(19) United States
(12) Patent Application Publication
(10) Pub. No.: US 2013/0251670 A1
(43) Pub. Date: Sep. 26, 2013

(54) Treatment of Macular Edema Utilizing Stem Cell and Conditioned Media Thereof

(71) Applicants: Neil H. Riordan, Trophy Club, TX (US); Jorge Paz Rodriguez, Panama (PA)

(72) Inventors: Neil H. Riordan, Trophy Club, TX (US); Jorge Paz Rodriguez, Panama (PA)

(73) Assignee: Aidan Products, Inc, Chandler, AZ (US)

(21) Appl. No.: 13/615,043

(22) Filed: Sep. 13, 2012

Related U.S. Application Data
(60) Provisional application No. 61/534,196, filed on Sep. 13, 2011.

(51) Int. Cl. A61K 35/48

(52) U.S. Cl. CPC A61K 35/51
USPC 424/85.2; 424/520; 424/529; 424/570; 424/574; 424/582; 424/583

(57) ABSTRACT

Described are methods of treating macular edema, as well as other complications of ocular inflammation, through the administration of various stem cell populations and conditioned media thereof. In one embodiment a patient suffering from macular edema is administered an intramuscular dose of media conditioned by placental mesenchymal stem cells at a sufficient concentration and frequency to elicit a reduction in ocular inflammation. Other embodiments of the invention teach intra-ocular, intravenous, sublingual, and oral means of delivery conditioned media from various stem cell populations that has been optimized for anti-inflammatory, regenerative, and immune modulatory properties.

### CROSS-REFERENCE TO RELATED APPLICATIONS

This application claims priority to co-pending U.S. Provisional Application Ser. No. 61/534,196 filed Sep. 13, 2011, which is expressly incorporated herein by reference in its entirety.

### FIELD OF THE INVENTION

The invention relates to the area of treatment compositions for ocular disorders. Particularly, the invention relates to generation of factors useful for the treatment of inflammatory ocular disorders. More particularly the invention relates to the area of utilizing stem cells and stem cell derived factors for the inhibition of macular edema.

### BACKGROUND

The classical definition of macular edema is accumulation of fluid in the outer plexiform layer and the inner nuclear layer of the eye, associated with swelling of the Müller cells of the retina. In the majority of cases macrular edema presents with localized expansion of the retinal extracellular space in the macular area. Macular edema is the manifestation of age-related macular degeneration, diabetic retinopathy and retinal vein occlusion, leading to loss of vision. Macular edema is commonly caused by perturbation of the blood-retinal barrier (BRB)[1,2]. Macular edema is the leading cause of blindness in young adult diabetics in developed countries, targeting 12% of type 1 and 28% of type 2 diabetic patients [3,4]. Macular edema is characterized by retinal thickening or the presence of hard exudates within a 1 disk diameter of the center of the macula. More advanced macular edema is called “clinically significant macular edema” in which: retinal thickening at or within 500 mm of the center of the macula; hard exudates are found at or within 500 mm of the center of the macula if associated with thickening of the adjacent retina; and a zone or zones of retinal thickening 1 disk area in size, at least part of which is within 1 disk diameter of the macular center, characterized by the retinal thickening of the macular area [5,6]. This condition is currently treated by control of glycemia, lipid levels and renal function [7,8]. However, which metabolic control is useful for patients with diabetic retinopathy, it has proven to be insufficient for patients suffering from macular edema once it occurs. Currently laser treatment has been the only intervention in this condition, however, it is inadequate in chronic cases. Currently there are some newly introduce treatments, such as intravitreal corticosteroids or anti-VEGF drugs [9-11], however only subsets of populations are eligible for these interventions. Additionally, these interventions are non-regenerative in nature and cannot accelerate healing once damage has already occurred. The invention provides for use of locally or systemically administered cells or trophic factors for stimulation of regeneration and/or inhibition of inflammation for addressing the issue of ocular disease in general and specifically inhibiting macular edema.

### SUMMARY

The invention describes the use of cells with regenerative potential and/or conditioned media from said cells for treatment of ocular disorders. Specifically, in one aspect of the invention, cells from the placental structure, such as mesenchymal-like cells derived from Wharton's Jelly, are used to generate a conditioned media product that can be administered systemically and elicit therapeutic activity in ocular diseases. The invention provides the unexpected findings that: A) conditioned media actually decreases ocular edema. This is counter-intuitive given that stem cell/regenerative cell conditioned media is generally considered pro-angiogeneic [12-17], and current treatments for ocular edema are anti-angiogenic[1,8], given that angiogenesis is believed to play a critical role in macular edema [1,9]. B) Systemic administration of relatively small amounts of conditioned media induces therapeutic benefit. In one aspect of the invention, the teachings in our previously filed patent application, U.S. Application No. 61/479,359 entitled Therapeutic Conditinoed Media, can be used for the treatment of ocular disorders, and particularly macular edema. This application along with all other references cited herein are expressly incorporated by reference herein in their entireties.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1: Full retina thickness map over time.

FIG. 2: Full retina thickness map images over time.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The invention teaches methods of generating a therapeutic product through growth of various cell populations with regenerative ability in a liquid media. Cell populations useful for the practice of the invention include: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. In one embodiment, the invention provides a means of creating a medicament useful for the treatment of ocular disorders of inflammatory, and/or autoimmune, and/or degenerative nature. Said conditions are treated using a medicament generated by culturing Wharton Jelly mesenchymal cells in a serum free media. Many types of media may be used and chosen by one of skill in the art. In one embodiment a media is selected from a group comprising of alpha MEM, DMEM, RPMI, Opti-MEM, IMEM, and AIM-V. Cells may be cultured in a variety of media for expansion that contain fetal calf serum, or other growth factors, however, for collection of therapeutic supernatant, in a preferred embodiment, the cells are transferred to a media substantially lacking serum. In some embodiments, the supernatant is administered directly into the patient in need of treatment. It is well known in the art that preparation of the supernatant before administration may be performed by various means, for example, said supernatant may be filter sterilized, or in some conditions concentrated. In a preferred embodiment, the supernatant is administrated intramuscularly in a volume of 0.5 to 1 ml per injection, with two injections per week. In this embodiment a concentration of 30 million Wharton Jelly mesenchymal cells are grown on a plastic surface for approximately 24 hours. Supernatant is harvested, filter sterilized, and stored for administration. Supernatant may alternatively be administered intra-ocularly. When treating a patient with macular edema, various parameters may be assessed specific to the condition. For example, daily eye examination and assessment of the anterior chamber inflammation using a laser flare meter during the first week post treatment may be performed. The eye examination at baseline and each follow up may consist of best corrected visual acuity, intra-ocular pressure using Goldman applanation tonometry, non dilated slit lamp examination with assessment of conjunctival and intraocular inflammation, filtering bleb morphology, size and function and filtering bleb leaks.

In other embodiments, the conditioned media is used as an active ingredient for the generation of a pharmaceutical formulation. This may comprise administration of the stem cell conditioned media therapeutic agent alone, but preferably comprise administration by way of known pharmaceutical formulations, including tablets, capsules or elixirs for oral administration, suppositories for rectal administration, sterile solutions or suspensions for parenteral or intramuscular administration, liposomal or encapsulated formulations, formulations wherein the therapeutic agent is alone or conjugated to a delivery agent or vehicle, and the like. It will be appreciated that therapeutic entities of the invention will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences (15.sup.th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol 52:238-311 (1998) and the citations therein for additional information related to excipients and carriers well known to pharmaceutical chemists. In one embodiment of the invention, one or more agents of the invention are nanoencapsulated into nanoparticles for delivery. The nanoencapsulation material may be biodegradable or nondegradable. The nanoencapsulation materials may be made of synthetic polymers, natural polymers, oligomers, or monomers. Synthetic polymers, oligomers, and monomers include those derived from polyalkyleneoxide precursor molecules, such as poly(ethylene oxide) (PEO), poly(ethylene glycol) (PEG) and copolymers with poly(propylene oxide) (PEG-co-PPO), poly (vinyl alcohol) (PVA), poly(vinylpyrrolidone) (PVP), poly(ethyloxazoline) (PEOX), polyaminoacids, and pseudopolyamino acids, and copolymers of these polymers. Sawhney et al., Macromolecules 26:581-587 (1993). Copolymers may also be formed with other water-soluble polymers or water insoluble polymers, provided that the conjugate is water soluble. An example of a water-soluble conjugate is a block copolymer of polyethylene glycol and polypropylene oxide, commercially available as a Pluronic™ surfactant (BASF). Natural polymers, oligomers and monomers include proteins, such as fibrinogen, fibrin, gelatin, collagen, elastin, zein, and albumin, whether produced from natural or recombinant sources, and polysaccharides, such as agarose, alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageen. These polymers are merely exemplary of the types of nanoencapsulation materials that can be utilized and are not intended to represent all the nanoencapsulation materials within which entrapment is possible. In one embodiment, the therapeutic agent is administered in a topical formulation. Topical formulations are useful in the treatment of conditions associated with dermal diseases. For example, topical administration of stem cell conditioned media may be performed for the treatment of psoriasis, scleroderma, or acne. Topical forms of administration may consist of, for example, aqueous and nonaqueous gels, creams, multiple emulsions, microemulsions, liposomes, ointments, aqueous and nonaqueous solutions, lotions, aerosols, skin patches, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Topical formulations of the invention may include a dermatologically acceptable carrier, e.g., a substance that is capable of delivering the other components of the formulation to the skin with acceptable application or absorption of those components by the skin. The carrier will typically include a solvent to dissolve or disperse the therapeutic agent, and, optionally one or more excipients or other vehicle ingredients. Carriers useful in accordance with the topical formulations of the present invention may include, by way of non-limiting example, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Topical formulations of the invention are prepared by mixing a compound of the invention with a topical carrier according to well-known methods in the art, for example, methods provided by standard reference texts e.g., Remington: The Science and Practice of Pharmacy, 1577-1591, 1672-1673, 866-885 (Alfonso R. Gennaro ed. 19th ed. 1995); and Ghosh et al., Transdermal and Topical Drug Delivery Systems (1997). In other embodiments, moisturizers or humectants, sunscreens, fragrances, dyes, and/or thickening agents such as paraffin, jojoba, PABA, and waxes, surfactants, occlusives, hygroscopic agents, emulsifiers, emollients, lipid-free cleansers, antioxidants and lipophilic agents, may be added to the topical formulations of the invention if desired. A topical formulation of the invention may be designed to be left on the skin and not washed shortly after application. Alternatively, the topical formulation may be designed to be rinsed off within a given amount of time after application.

In one embodiment of the invention, potency of the conditioned media product may be quantified by use of assessing protein production. Such assays are well-known to one of skill in the art. Following the teachings of Jiao et al. [20], production of IL-10 may be quantified. For quantification of anti-inflammatory activity, the term “inflammation” will be understood by those skilled in the art to include any condition characterized by a localized or a systemic protective response, which may be elicited by physical trauma, infection, chronic diseases, such as those mentioned above, and/or chemical and/or physiological reactions to external stimuli (e.g., as part of an allergic response). Any such response, which may serve to destroy, dilute or sequester both the injurious agent and the injured tissue, may be manifested by, for example, heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or condition per se, any condition that has an inflammatory component associated with it, and/or any condition characterized by inflammation as a symptom, including, inter alia, acute, chronic, ulcerative, specific, allergic and necrotic inflammation, and other forms of inflammation known to those skilled in the art. The term thus also includes, for the purposes of this invention, inflammatory pain and/or fever caused by inflammation.

In one embodiment, stem cell conditioned media is used in combination with an immune suppressive agent to augment its activity. While stem cell conditioned media may be used alone for treatment and/or maintenance of disease remission, in some embodiments coadministration with an immune suppressive agent may be required. Additionally, an immune suppressive agent may be useful for “induction therapy”. Depending on disease and response desired, it will be known to one of skill in the art to choose from various immune suppressive agents. For example, some immune suppressive agents, such as anti-CD52 antibodies may be used when a systemic depletion of T and B cells is desired, whereas agents that concurrently stimulate T regulatory cell activity, such as Rapamycin, may be desired in other cases. The skilled practitioner is guided to several agents that are known in the art for causing immune suppression, which include cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-.alpha. inhibitors, TNF-.alpha. sequestration agents, and methotrexate. More specifically, anti-inflammatory agents may comprise one or more of, e.g., anti-TNF-.alpha., lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.- acetamidoc aproic acid, s-adenosylmethionine, 3-amino-4-hydroxybutyric .acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, etc.

When selecting stem cells for use in the practice of the current invention, several factors must be taken into consideration, such as: ability for ex vivo expansion without loss of ability to secrete therapeutic factors, ease of extraction, and general potency of activity. Ex vivo expansion ability of stem cells can be measured using typical proliferation and colony assays known to one skilled in the art, while identification of therapeutic activity depends on functional assays that test biological activities such as: ability to support endothelial function, ability to protect neurons from degeneration/atrophy, and induce proliferation of endogenous stem cells. Assessment of therapeutic activity can also be performed using surrogate assays which detect markers associated with a specific therapeutic activity. Such markers include CD34 or CD133, which are associated with stem cell activity and ability to support angiogenesis [21]. Other assays useful for identifying therapeutic activity of stem cell populations for use with the current invention include evaluation of production of factors associated with the therapeutic activity desired. For example, identification and quantification of production of FGF, VEGF, angiopoietin, or other such angiogenic molecules may be used to serve as a guide for approximating and quantifying growth factor/anti-apoptotic factors elaborated by said cells into culture media [22].

For quantification of effects that stem cells have on conditioned media, and therefore a quantification of the potency of conditioned media, one needs to first decide the therapeutic indication sought. If one seeks to utilize conditioned media for immune suppression, one may assess levels of immune modulatory components in said conditioned media. Examples of soluble immune suppressive factors include: IL-4 [23], IL-10 [24], IL-13 [25], TGF-b [26], soluble TNF-receptor [27], and IL-1 receptor agonist [28]. Membrane-bound immunoinhibitor molecules that may be shed by stem cells and therefore another marker for quantification of specific therapeutic properties: HLA-G [29], FasL [30], PD-1L [31], Decay Accelerating Factor [32], and membrane-associated TGF-b [33]. Enzymes whose biological activity causes alteration in supernatant composition to possess immune suppressive activities include indolamine 2,3 dioxygenase [34] and arginase type II [35]. In order to optimize desired immune suppressive ability, a wide variety of assays are known in the art, including mixed lymphocyte culture, ability to generate T regulatory cells in vitro, and ability to inhibit natural killer or CD8 cell cytotoxicity. In situations where increased angiogenic potential of said conditioned media therapeutic product is desired, assessment of proteins associated with stimulation of angiogenesis may be performed. These include VEGF[36], FGF1 [37], FGF2 [38], FGF4 [39], FrzA [40], and angiopoietin [41]. In some situations the cells in contact with media that generate conditioned media may be transfected with genes to allow for enhanced cellular viability, anti-apoptotic genes suitable for transfection may include bcl-2 [42], bcl-xl [43], and members of the XIAP family [44]. Alternatively it may be desired to increase the proliferative lifespan of said mesenchymal stem cells through transfection with enzymes associated with anti-senescence activity. Said enzymes may include telomerase or histone deacetylases.

In one embodiment mesenchymal cells are generated through culture and subsequently culture media is used for generation of a therapeutic composition. Said therapeutic composition is preferably generated in a medium that is free from human or animal products, with said medium also lacking phenol red. For extraction and growth of mesenchymal stem cells, the skilled practitioner of the invention is referred to examples known in the literature, which include U.S. Pat. No. 5,486,359 describing methods for culturing such and expanding mesenchymal stem cells, as well as providing antibodies for use in detection and isolation. Additionally, U.S. Pat. No. 5,942,225 teaches culture techniques and additives for differentiation of such stem cells which can be used in the context of the present invention to produce increased numbers of cells with ability to secrete agents that possess angio genic activities. Although U.S. Pat. No. 6,387,369 teaches use of mesenchymal stem cells for regeneration of cardiac tissue, we believe that in accordance with published literature [45, 46] stem cells generated through these means are actually angiogenically potent and therefore may be utilized in the context of the current invention. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1 [47], quantification of said growth factors may be useful in standardizing doses in the preparation of said stem cell conditioned media therapeutic product.

Historically, MSC are obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue [48], placenta and Wharton's Jelly [49, 50], scalp tissue [51] and cord blood [52]. While mesenchymal stem cells generated from bone marrow, cord blood, and adipose tissue appear to possess similar morphology and phenotype, ability to induce colony formation appears to be highest using stem cells from adipose tissue and interestingly in contrast to bone marrow and adipose derived mesenchymal cells, only the cord blood derived cells lacked ability to undergo adipocyte differentiation. Within the context of the current invention, our data suggests that conditioned media generated using Wharton's Jelly as a source of cells possesses unique characteristics in contrast to adipose-derived stem cells. It is also known that the proliferative potential appears to be the highest with cord blood mesenchymal stem cells which were capable of expansion to approximately 20 times, whereas cord blood cells expanded an average of 8 times and bone marrow derived cells expanded 5 times [53]. Accordingly, one skilled in the art will understand that mesenchymal stem cells for use with the present invention may be selected upon individual patient characteristics and the end result sought.

For use in the context of the present invention, embryonic stem cells possess certain desirable properties, which include unique “early” growth factor production profile. It is believed in the art that many of the therapeutic effects of ES cell administration are mediated by paracrine factors. This is promising since therapeutic use of ES cells themselves is limited by formation of teratomas [54]. Another embodiment of the current invention is the use of embryonic stem cell supernatant as a therapeutic product. Specific embodiments include identification of substantially purified fractions of said supernatant capable of inducing endothelial cell proliferation, smooth muscle regeneration, and/or neuronal cell proliferation/survival, and/or anti-inflammatory activity, and/or stimulation of endogenous reparative processes. Identification of such therapeutically active fractions may be performed using methods commonly known to one skilled in the art, and includes separation by molecular weight, charge, affinity towards substrates and other physico-chemical properties. In one particular embodiment, supernatant of embryonic stem cell cultures is harvested substantially free from cellular contamination by use of centrifugation or filtration. Supernatant may be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of embryonic stem cell supernatant may be passed through each cartridge before elution. After washing the cartridges material adsorbed is eluted with 3 ml methanol, evaporated under a stream of nitrogen, redissolved in a small volume of methanol, and stored at 4.degree. C. Before testing the eluate for activity in vitro, the methanol is evaporated under nitrogen and replaced by culture medium. Said C18 cartridges are used to adsorb small hydrophobic molecules from the embryonic stem cell culture supernatant, and allows for the elimination of salts and other polar contaminants. It may, however be desired to use other adsorption means in order to purify certain compounds from the embryonic stem cell supernatant. Said concentrated supernatant may be assessed directly for biological activities useful for the practice of this invention, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Embryonic stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically. For the practice of the invention, the practitioner is referred to the numerous methods of generating embryonic stem cells that are known in the art. Patents describing the generation of embryonic stem cells include U.S. Pat. No. 6,506,574 to Rambhatla, 6,200,806 to Thomson, 6,432,711 to Dinsmore, and 5,670,372 to Hogan.

In one embodiment of the invention, embryonic stem cells are differentiated into endothelial progenitor cells in vitro, followed by administration of conditioned media from these cells to a patient in need of therapy at a concentration and frequency sufficient to induce a therapeutic response. Differentiation into endothelial progenitors may be performed by several means known in the art [55]. One such means includes generation of embryoid bodies through growing human embryonic stem cells in a suspension culture. Said embryoid bodies are subsequently dissociated and cells expressing endothelial progenitor markers are purified [56]. Purification of endothelial cells from embryoid bodies can be performed using, of example, selection for PECAM-1 expressing cells. Another alternative method of generating endothelial progenitors for use in generation of conditioned media from embryonic stem cells involves removing media from embryonic stem cells a period of time after said embryonic stem cells are plated and replacing said media with a media containing endothelial-differentiating factors. For example, after plating of embryonic stem cells for a period between 6 and 48 hours, but more preferably between 20 and 24 hours, the original media in which embryonic stem cells were cultured is washed off the cells and endothelial cell basal medium-2 (EBM2), with 5% fetal calf serum, VEGF, bFGF, IGF-1, EGF, and ascorbic acid is added to the cells. This combination is commercially available (EGM2-MV Bullet Kit; Clonetics/BioWhittaker, Walkersville, Md.). By culturing the embryonic stem cells for 20-30 days in the EGM2 medium, with changing of media every 3 to 5 days, a population of endothelial progenitors can be obtained. For such cells to be useful in the practice of the present invention, functionality of growth factors produced by said endothelial precursors, and their differentiated progeny must be assessed. Methods of assessing stimulation of angiogenesis are well known in the art [57].

For the practice of the invention supernatants generated by culture with cells may be administered to the patient in an injection solution, which may be saline, mixtures of autologous plasma together with saline, or various concentrations of albumin with saline. Ideally pH of the injection solution is from about 6.4 to about 8.3, optimally 7.4. Excipients may be used to bring the solution to isotonicity such as, 4.5% mannitol or 0.9% sodium chloride, pH buffers with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. Injection can be performed systemically, or more specifically, via routes of administration selected from; a) orally; b) intravenously; c) intramuscularly; d) intraperitoneally; e) intrathecally; f) alimentarily; g) intraspinally; h) intra-articularly; i) intra-joint; j) subcutaneously; k) buccally; 1) vaginally; m) rectally; n) dermally; o) transdermally; p) ophthalmically; q) auricularly; r) mucosally; s) nasally; t) tracheally; u) bronchially; v) sublingually; w) intranodally; x) by any parenteral route; and y) via inhalation

In one particular method, cord blood cells are used for generation of conditioned media, said cord blood is collected from fresh placenta and mononuclear cells are purified by centrifugation using a density gradient such as Ficoll or Percoll, in another method cord blood mononuclear cells are isolated from contaminating erythrocytes and granulocytes by the Hetastarch with a 6% (wt/vol) hydroxyethyl starch gradient. Cells are subsequently washed to remove contaminating debris, assessed for viability, and admixed with culture media to generate a conditioned media. As described within this application, conditioned media is ideally generated for practice within the current invention by a 24 hour culture, however one of skill in the art may identify other time points without deviated from the spirit of the invention. In another embodiment of the invention, cord blood stem cells are fractionated and the fraction with enhanced therapeutic activity is administered to the patient. Enrichment of cells with therapeutic activity may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the invention, it will be useful to select cord blood stem cells with a higher proliferative ability, whereas in other situations, a lower proliferative ability may be desired. In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for ED, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

Certain desired activities can be endowed onto said cord blood stem cells prior to using as a source of cells for generation of conditioned media. In one specific embodiment cord blood cells may be “activated” ex vivo by a brief culture in hypoxic conditions in order to upregulate nuclear translocation of the HIF-1 transcription factor and endow said cord blood cells with enhanced production of angiogenic growth factors. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, can be performed during optimization of conditioned media production. In addition to induction of hypoxia, other therapeutic properties can be endowed unto cord blood stem cells through treatment ex vivo with factors such as de-differentiating compounds, proliferation inducing compounds, or compounds known to endow and/or enhance cord blood cells to possess properties useful for the practice of the current invention. In one embodiment cord blood cells are cultured with an inhibitor of the enzyme GSK-3 in order to enhance expansion of cells with pluripotent characteristics while not increasing the rate of differentiation. In another embodiment, cord blood cells are cultured in the presence of a DNA methyltransferase inhibitor such as 5-azacytidine in order to endow a “de-differentiation” effect. In another embodiment cord blood cells are cultured in the presence of a differentiation agent that skews said cord blood stem cells to generate enhance numbers of cells which are useful for generation of conditioned media.

In contrast to cord blood stem cells, placental stem cells may be purified directly from placental tissues, said tissues including the chorion, amnion, and villous stroma [49, 58]. In another embodiment of the invention, placental tissue is mechanically degraded in a sterile manner and treated with enzymes to allow dissociation of the cells from the extracellular matrix. Such enzymes include, but not restricted to trypsin, chymotrypsin, collagenases, elastase and/or hylauronidase. Suspension of placental cells are subsequently washed, assessed for viability, and may either be used directly for the practice of the invention. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations it will be desirable to expand cells before use for generation of conditioned media. Expansion can be performed by culture ex vivo with specific growth factors [59, 60]. The various embodiments of the invention described above for cord blood and embryonic stem cells can also be applied for placental stem cells.

Bone marrow stem cells may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest” [61]. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass), or the Fresenius AS 104 and the Fresenius AS TEC 104 (Fresenius, Bad Homburg, Germany) separators. Additionally, ex vivo expansion and/or selection may also be utilized for augmentation of desired biological properties for use in creation of conditioned media. The various embodiments of the invention described above for cord blood and embryonic stem cells can also be applied for bone marrow stem cells.

Amniotic fluid is routinely collected during amniocentesis procedures. One method of practicing the current invention is utilizing amniotic fluid derived stem cells for generation of conditioned media. In one embodiment amniotic fluid mononuclear cells are utilized therapeutically in an unpurified manner or heterogeneous manner. Said amniotic fluid stem cells are used to endow therapeutic properties on media. In other embodiments amniotic fluid stem cells are substantially purified based on expression of markers such as S SEA-3, SSEA4, Tra-1-60, Tra-1-81 and Tra-2-54, and subsequently administered. In other embodiments cells are cultured, as described in US patent application #20050054093, expanded, and subsequently used for production of conditioned media. Amniotic stem cells are described in the following references [62-64]. One particular aspect of amniotic stem cells that makes them amenable for use in practicing certain aspects of the current invention is their bi-phenotypic profile as being both mesenchymal and neural progenitors [65]. This property is useful for treatment of patients with conditions associated with neurological dysfunction.

Stem cells committed to the neuronal lineage, or neuronal progenitor cells, are used in the practice of some specific embodiments of the invention. Said cells may be generated by differentiation of embryonic stem cells, may be freshly isolated from fetal tissue (i.e. mesencephalic), may be generated by transdifferentiation, or by reprogramming of a cell. Neuronal progenitors are selected by use of markers such as polysialyated N-CAM, N-CAM, A2B5, nestin and vimentin. The various embodiments of the invention described above for cord blood and embryonic stem cells can also be applied for neuronal stem cells.

A wide variety of stem cells are known to circulate in the periphery. These include multipotent, pluripotent, and committed stem cells. In some embodiments of the invention mobilization of stem cells is induced in order to increase the number of circulating stem cells, so that harvesting efficiency is increased. Said mobilization allows for harvest of cells with desired properties for practice of the invention without the need to perform bone marrow puncture. A variety of methods to induce mobilization are known. Methods such as administration of cytotoxic chemotherapy, for example, cyclophosphamide or 5-fluoruracil are effective but not preferred in the context of the current invention due to relatively unacceptable adverse events profile. Suitable agents useful for mobilization include: granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), thrombopoietin (TPO), interleukin-11 (IL-11), insulin-like growth factor-1 (IGF-1), megakaryocyte growth and development factor (MGDF), nerve growth factor (NGF), hyperbaric oxygen, and 3-hydroxy-3-methyl glutaryl coenzyme A (HMG CoA) reductase inhibitors. The various embodiments of the invention described above for cord blood and embryonic stem cells can also be applied for circulating peripheral blood stem cells.

Adipose derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently washed and cultured for 24 hours in DMEM media.

### Example 1

### Production of Conditioned Media

Human umbilical cords were obtained from healthy mothers in our hospital after they gave their informed consent. Umbilical cords were processed within 4 h and stored at 4° C. in sterile saline until use. The cords were rinsed several times in sterile phosphate-buffered saline (PBS) to remove blood components and cut into small pieces (2-3 cm). Cord vessels (2 arteries and 1 vein) were removed to avoid endothelial cell contamination. The Wharton's jelly parts of the cord were cut into pieces 0.5-1 cm3 and placed directly into 75-cm2 flasks for culture expansion in low-glucose Dulbecco's modified Eagle's medium (LG-DMEM) containing 10% fetal bovine serum (FBS), 100 U/ml penicillin/streptomycin at 37° C., and 5% (v/v) CO2. Cells were detached with 0.05% trypsin-EDTA and reseeded in new culture flasks. When cells reached 75% confluence, they were washed with PBS and serum free, phenol-free DMEM media was added for 24 hours to a total of approximately 30 million cells. Supernatant was collected, filter sterilized with a 0.2 micron filter and either frozen or lyophilized for further use.

### Example 2

### Treatment of Macular Edema Patient

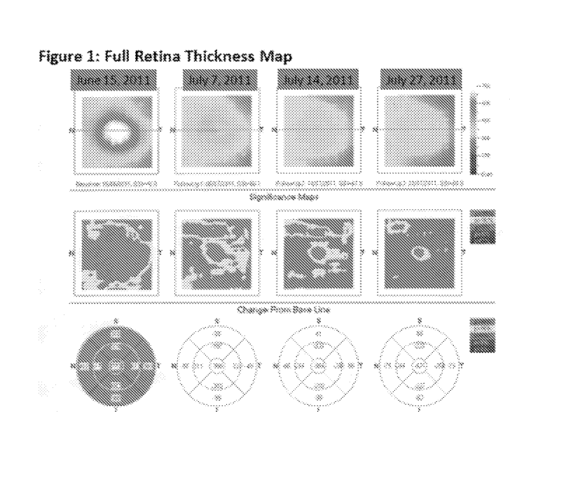

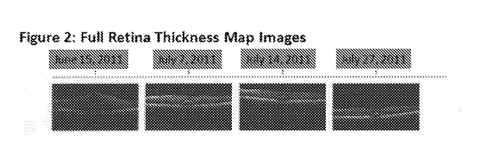

A 33 years old patient suffering from macular edema was administered the composition described in Example 1 two times a week for a period of four weeks intramuscularly. The patient was examined pre-treatment and post-treatment with the Optovue ultra-high speed, high resolution OCT retina scanner used for retina imaging and analysis. As seen in FIGS. 1 and 2, a significant decrease in macular edema and near-normalization of the retina was observed after treatment.

### REFERENCES

1. Guex-Crosier, Y., The pathogenesis and clinical presentation of macular edema in inflammatory diseases. Documenta ophthalmologica. Advances in ophthalmology, 1999. 97(3-4): p. 297-309.
2. Scholl, S., J. Kirchhof, and A. J. Augustin, Pathophysiology of macular edema. Ophthalmologica. Journal international d′ ophtalmologie. International journal of ophthalmology. Zeitschrift fur Augenheilkunde, 2010. 224 Suppl 1: p. 8-15.
3. Romero-Aroca, P., Managing diabetic macular edema: The leading cause of diabetes blindness. World journal of diabetes, 2011. 2(6): p. 98-104.
4. Klein, R., et al., The Wisconsin Epidemiologic Study of Diabetic Retinopathy. XV. The long-term incidence of macular edema. Ophthalmology, 1995. 102(1): p. 7-16.
5. Grading diabetic retinopathy from stereoscopic color fundus photographs—an extension of the modified Airlie House classification. ETDRS report number 10. Early Treatment Diabetic Retinopathy Study Research Group. Ophthalmology, 1991. 98(5 Suppl): p. 786-806.
6. Classification of diabetic retinopathy from fluorescein angiograms. ETDRS report number 11. Early Treatment Diabetic Retinopathy Study Research Group. Ophthalmology, 1991. 98(5 Suppl): p. 807-22.
7. Benarous, R., et al., Differential Association of Serum Lipids with Diabetic Retinopathy and Diabetic Macular Edema. Investigative ophthalmology & visual science, 2011.
8. Romero-Aroca, P., et al., Ten-year incidence of diabetic retinopathy and macular edema. Risk factors in a sample of type 1 diabetes patients. Diabetes research and clinical practice, 2011.
9. Wirostko, B., et al., Patient preferences in the treatment of diabetic retinopathy. Patient preference and adherence, 2011. 5: p. 229-37.
10. Channa, R., M. Smith, and P. A. Campochiaro, Treatment of macular edema due to retinal vein occlusions. Clinical ophthalmology, 2011. 5: p. 705-13.
11. Sun, J. K., Intravitreal anti-VEGF therapy with prompt or deferred laser compared with steroid with prompt laser and prompt laser alone for treatment of diabetic macular edema. Current diabetes reports, 2011. 11(4): p. 227-9.
12. Sahoo, S., et al., Exosomes From Human CD34+Stem Cells Mediate Their Proangiogenic Paracrine Activity. Circulation research, 2011.
13. Konig, J. M., et al., Amnion-derived mesenchymal stromal cells show angiogenic properties but resist differentiation into mature endothelial cells. Stem cells and development, 2011.
14. Oskowitz, A., et al., Serum-deprived human multipotent mesenchymal stromal cells (MSCs) are highly angiogenic. Stem cell research, 2011. 6(3): p. 215-25.
15. Timmers, L., et al., Human mesenchymal stem cell-conditioned medium improves cardiac function following myocardial infarction. Stem cell research, 2011. 6(3): p. 206-14.
16. Teodelinda, M., et al., Amniotic liquid derived stem cells as reservoir of secreted angiogenic factors capable of stimulating neo-arteriogenesis in an ischemic model. Biomaterials, 2011. 32(15): p. 3689-99.
17. Rasmussen, J. G., et al., Prolonged hypoxic culture and trypsinization increase the pro-angiogenic potential of human adipose tissue-derived stem cells. Cytotherapy, 2011. 13(3): p. 318-28.
18. Sohn, H. J., et al., Changes in Aqueous Concentrations of Various Cytokines After Intravitreal Triamcinolone vs Bevacizumab for Diabetic Macular Edema. American journal of ophthalmology, 2011.
19. Liu, X., et al., Endothelial progenitor cells (EPCs) mobilized and activated by neurotrophic factors may contribute to pathologic neovascularization in diabetic retinopathy. The American journal of pathology, 2010. 176(1): p. 504-15.
20. Jiao, J., et al., A mesenchymal stem cell potency assay. Methods in molecular biology, 2011. 677: p. 221-31.
21. Higashi, Y., et al., Oxidative stress, endothelial function and angiogenesis induced by cell therapy and gene therapy. Curr Pharm Biotechnol, 2006. 7(2): p. 109-16.
22. Tonnesen, M. G., X. Feng, and R. A. Clark, Angiogenesis in wound healing. J Investig Dermatol Symp Proc, 2000. 5(1): p. 40-6.
23. Jansen, J. H., et al., Interleukin-4. A regulatory protein. Blut, 1990. 60(5): p. 269-74.
24. Zhou, X., et al., Boosting interleukin-10 production: therapeutic effects and mechanisms. Curr Drug Targets Immune Endocr Metabol Disord, 2005. 5(4): p. 465-75.
25. Mentink-Kane, M. M. and T. A. Wynn, Opposing roles for IL-13 and IL-13 receptor alpha 2 in health and disease. Immunol Rev, 2004. 202: p. 191-202.
26. Kriegel, M. A., et al., Transforming growth factor-beta: recent advances on its role in immune tolerance. Curr Rheumatol Rep, 2006. 8(2): p. 138-44.
27. Fernandez-Botran, R., F. A. Crespo, and X. Sun, Soluble cytokine receptors in biological therapy. Expert Opin Biol Ther, 2002. 2(6): p. 585-605.
28. Dayer, J. M., Evidence for the biological modulation of IL-1 activity: the role of IL-1Ra. Clin Exp Rheumatol, 2002. 20(5 Suppl 27): p. S14-20.
29. Rouas-Freiss, N., et al., HLA-G proteins in cancer: do they provide tumor cells with an escape mechanism? Cancer Res, 2005. 65(22): p. 10139-44.
30. Bohana-Kashtan, O. and C. I. Civin, Fas ligand as a tool for immunosuppression and generation of immune tolerance. Stem Cells, 2004. 22(6): p. 908-24.
31. Okazaki, T. and T. Honjo, The PD-1-PD-L pathway in immunological tolerance. Trends Immunol, 2006. 27(4): p. 195-201.
32. Longhi, M. P., et al., Holding T cells in check—a new role for complement regulators? Trends Immunol, 2006. 27(2): p. 102-8.
33. Wahl, S. M., J. M. Orenstein, and W. Chen, TGF-beta influences the life and death decisions of T lymphocytes. Cytokine Growth Factor Rev, 2000. 11(1-2): p. 71-9.
34. Mellor, A. L. and D. H. Munn, IDO expression by dendritic cells: tolerance and tryptophan catabolism. Nat Rev Immunol, 2004. 4(10): p. 762-74.
35. Serafini, P., I. Bonello, and V. Bronte, Myeloid suppressor cells in cancer: recruitment, phenotype, properties, and mechanisms of immune suppression. Semin Cancer Biol, 2006. 16(1): p. 53-65.
36. Matsumoto, R., et al., Vascular endothelial growth factor-expressing mesenchymal stem cell transplantation for the treatment of acute myocardial infarction. Arterioscler Thromb Vasc Biol, 2005. 25(6): p. 1168-73.
37. Klein, S., M. Roghani, and D. B. Rifkin, Fibroblast growth factors as angiogenesis factors: new insights into their mechanism of action. Exs, 1997. 79: p. 159-92.
38. Chen, C. H., et al., Fibroblast growth factor 2: from laboratory evidence to clinical application. Curr Vasc Pharmacol, 2004. 2(1): p. 33-43.
39. Grines, C., et al., Angiogenic gene therapy with adenovirus 5 fibroblast growth factor-4 (Ad5FGF-4): a new option for the treatment of coronary artery disease. Am J Cardiol, 2003. 92(9B): p. 24N-31N.
40. Dufourcq, P., et al., FrzA, a secreted frizzled related protein, induced angiogenic response. Circulation, 2002. 106(24): p. 3097-103.
41. Morisada, T., et al., Angiopoietins and angiopoietin-like proteins in angiogenesis. Endothelium, 2006. 13(2): p. 71-9.
42. Murphy, E., K. Imahashi, and C. Steenbergen, Bc1-2 regulation of mitochondrial energetics. Trends Cardiovasc Med, 2005. 15(8): p. 283-90.
43. Harada, H. and S. Grant, Apoptosis regulators. Rev Clin Exp Hematol, 2003. 7(2): p. 117-38.
44. Guegan, C., et al., PTD-XIAP protects against cerebral ischemia by anti-apoptotic and transcriptional regulatory mechanisms. Neurobiol Dis, 2006. 22(1): p. 177-86.
45. Caplan, A. I. and J. E. Dennis, Mesenchymal stem cells as trophic mediators. J Cell Biochem, 2006.
46. Shyu, K. G., et al., Mesenchymal stem cells are superior to angiogenic growth factor genes for improving myocardial performance in the mouse model of acute myocardial infarction. J Biomed Sci, 2006. 13(1): p. 47-58.
47. Nagaya, N., et al., Transplantation of mesenchymal stem cells improves cardiac function in a rat model of dilated cardiomyopathy. Circulation, 2005. 112(8): p. 1128-35.
48. Knippenberg, M., et al., Adipose tissue-derived mesenchymal stem cells acquire bone cell-like responsiveness to fluid shear stress on osteogenic stimulation. Tissue Eng, 2005. 11(11-12): p. 1780-8.
49. Portmann-Lanz, C. B., et al., Placental mesenchymal stem cells as potential autologous graft for pre- and perinatal neuroregeneration. Am J Obstet Gynecol, 2006. 194(3): p. 664-73.
50. Zhang, X., et al., Mesenchymal progenitor cells derived from chorionic villi of human placenta for cartilage tissue engineering. Biochem Biophys Res Commun, 2006. 340(3): p. 944-52.
51. Shih, D. T., et al., Isolation and characterization of neurogenic mesenchymal stem cells in human scalp tissue. Stem Cells, 2005. 23(7): p. 1012-20.
52. Kadivar, M., et al., In vitro cardiomyogenic potential of human umbilical vein-derived mesenchymal stem cells. Biochem Biophys Res Commun, 2006. 340(2): p. 639-47.
53. Kern, S., et al., Comparative Analysis of Mesenchymal Stem Cells from Bone Marrow, Umbilical Cord Blood or Adipose Tissue. Stem Cells, 2006.
54. Vogel, G., Cell biology. Ready or not? Human ES cells head toward the clinic. Science, 2005. 308(5728): p. 1534-8.
55. Levenberg, S., Engineering blood vessels from stem cells: recent advances and applications. Curr Opin Biotechnol, 2005. 16(5): p. 516-23.
56. Trounson, A., The production and directed differentiation of human embryonic stem cells. Endocr Rev, 2006. 27(2): p. 208-19.
57. Gerecht-Nir, S., et al., Human embryonic stem cells as an in vitro model for human vascular development and the induction of vascular differentiation. Lab Invest, 2003. 83(12): p. 1811-20.
58. Demir, R., et al., Classification of human placental stem villi: review of structural and functional aspects. Microsc Res Tech, 1997. 38(1-2): p. 29-41.
59. Mohamed, A. A., et al., Ex vivo expansion of stem cells: defining optimum conditions using various cytokines. Lab Hematol, 2006. 12(2): p. 86-93.
60. Kashiwakura, I. and T. A. Takahashi, Fibroblast growth factor and ex vivo expansion of hematopoietic progenitor cells. Leuk Lymphoma, 2005. 46(3): p. 329-33.
61. Mineishi, S., [Immunobiology of mini-transplant]. Nippon Rinsho, 2003. 61(9): p. 1489-94.
62. Bossolasco, P., et al., Molecular and phenotypic characterization of human amniotic fluid cells and their differentiation potential. Cell Res, 2006. 16(4): p. 329-36.
63. Sartore, S., et al., Amniotic mesenchymal cells autotransplanted in a porcine model of cardiac ischemia do not differentiate to cardiogenic phenotypes. Eur J Cardiothorac Surg, 2005. 28(5): p. 677-84.
64. Prusa, A. R., et al., Neurogenic cells in human amniotic fluid. Am J Obstet Gynecol, 2004. 191(1): p. 309-14.
65. Tsai, M. S., et al., Clonal amniotic fluid-derived stem cells express characteristics of both mesenchymal and neural stem cells. Biol Reprod, 2006. 74(3): p. 545-51.

1. A method of treating an ocular disorder comprising the steps of: a) extracting a cell possessing regenerative activity; b) contacting said cell with a liquid media; c) extracting liquid media; and d) administering said media to a patient in need thereof.

2. The method of claim 1, wherein said ocular disorder is selected from a group of disorders comprising: a) macular degeneration; b) diabetic retinopathy; c) dry eye syndrome; and d) macular edema.

3. The method of claim 1, wherein said ocular disorder is selected from a group comprising of: dry disorders of cornea and conjunctiva; b) disorders of the keratoconjunctival epithelium; c) syndrome with decrease in tear secretion; d) xerophthalmia; e) dry eye due to aging; f) ophthalmopathy in Stevens-Johnson syndrome; g) ophthalmopathy in Sjogren's syndrome; h) keratoconjunctival ulcer; i) dryness in wearing of contact lens; and j) surgery/lasix associated eye dryness.

4. The method of claim 3, wherein said ocular disorder is treated by topically applying said liquid media extracted from claim 1.

5. The method of claim 1, wherein said cell possessing regenerative activity is selected from a group of cells comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

6. The method of claim 5, wherein said embryonic stem cells are totipotent and express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

7. The method of claim 5, wherein said cord blood stem cells are multipotent and capable of differentiating into endothelial, smooth muscle, and neuronal cells.

8. The method of claim 7, wherein said cord blood stem cells are identified based on expression of one or more antigens selected from a group comprising: SSEA-3, SSEA-4, CD9, CD34, c-kit, OCT-4, Nanog, and CXCR-4

9. The method of claim 8, wherein said cord blood stem cells do not express one or more markers selected from a group comprising of: CD3, CD34, CD45, and CD11b.

10. The method of claim 13, wherein said placental stem cells are isolated from the placental structure.

11. The method of claim 10, wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

12. The method of claim 5, wherein said bone marrow stem cells comprise of bone marrow mononuclear cells.

13. The method of claim 12, wherein said bone marrow stem cells are selected based on the ability to differentiate into one or more of the following cell types: endothelial cells, smooth muscle cells, and neuronal cells.

14. The method of claim 13, wherein said bone marrow stem cells are selected based on expression of one or more of the following antigens: CD34, c-kit, flk-1, Stro-1, CD105, CD73, CD31, CD146, vascular endothelial-cadherin, CD133 and CXCR-4.

15. The method of claim 10, wherein said placental stem cells are isolated from the Wharton's Jelly.

16. The method of claim 10, wherein said placental stem cells are mesenchymal in morphology.

17. The method of claim 16, wherein said placental cell expresses one or more cytokines associated with a regenerative activities.

18. The method of claim 17, wherein said cytokines associated with regenerative activity as selected from a group comprising of CS-6, IL-6, IL-8, SDF-1, CXCL5, VEGF, CXCL6, COL4A4, MMP13, CYP7B1, ADAMDEC1, SLC6A1, CXCL1, PF4V1, CXCL3, CH25H, SFRP2, MMP1, DARC, HCK, bFGF, ERC2, CLIC6, and BCL8 in a 75% confluent culture.

19. The method of claim 18, wherein said placental stem cells exhibit expression of about 0.0-2,200.0 pg/ml of each of the one or more cytokines in a 75% confluent culture.

20. The method of claim 18, wherein said placental cell exhibits expression of between 200.0-1900.0 pg/ml of IL-6 in a 75% confluent culture.

21. The method of claim 18, wherein, said cell expresses between 350.0-2,000.0 pg/ml of bFGF in a 75% confluent culture.

22. The method of claim 18, wherein said cell expresses between 500.0-2,500.0 pg/ml of VEGF in a 75% confluent culture.

23. The method of claim 18, wherein said cell expresses between 140.0-1,500.0 pg/ml of SDF-1 in a 75% confluent culture.

24. The method of claim 1, wherein said cell is an immortalized cell with regenerative activity.

25. The method of claim 24, wherein said cell population is immortalized by means selected from a group comprising of; a) transfection with an oncogene; b) transfection telomerase; and c) transfection with a combination of an oncogene and telomerase.

26. The method of claim 25, wherein said cell population is immortalized by means of transfection with an oncogene selected from a group of oncogenes comprising of: a) abl; b) Af4/hrx; c) akt-2; d) alk; e) alk/npm; f) aml1; g) aml1/mtg8; h) bcl-2, 3, 6; i) bcr/abl; j) c-myc; k) dbl; l) dek/can; m) E2A/pbx1; n) egfr; o) enl/hrx; p) erg/TLS; q) erbB; r) erbB-2; s) ets-1; t) ews/fli-1; u) fms; v) fos; w) fps; x) gli; y) gsp; z) gsp; aa) HER2/new; ab) hox11; ac) hst; ad) IL-3; ae) int-2; af) jun; ag) kit; ah) KS3; ai) K-sam; aj) Lbc; ak) lck; al) Imol, Imo-2; am) L-myc; an) lyl-1; ao) lyt-10; ap) lyt-10/C alpha 1; aq) mas; ar) mdm-2; as) mll; at) mos; au) mtg8/am11; av) myb; aw) MYH11; ax) new; ay) N-myc; az) ost; ba) pax-5; bb) pbx1/E2a; bc) pim-1; bd) PRAD-1; be) raf; bf) RAR/PML; bg) Ras H, K, N; bh) rel/nrg; bi) ret; bj) rhom1, rhom2; bk) ros; bl) ski; bm) sis; bn) set/can; bo) src; bp) Tal1, tal2; bq) tan-1; br) Tiam1; bs) TSC2; and bt) trk.

27. The method of claim 5, wherein said adipose stem cell expresses markers selected from a group comprising of: CD13, CD29, CD44, CD63, CD73, CD90, CD166, Aldehyde dehydrogenase (ALDH), and ABCG2.

28. The method of claim 26, wherein said adipose tissue derived stem cells are a population of purified mononuclear cells extracted from adipose tissue capable of proliferating in culture for more than 1 month.

29. The method of claim 5, wherein said side population cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

30. The method of claim 1, wherein said liquid media is selected from a group of media useful for maintaining cell viability in culture consisting of a group comprising of: a) alpha MEM; b) DMEM; c) RPMI; d) Opti-MEM; e) IMEM; and f) AIM-V media.

31. The method of claim 1, wherein said cells are expanded in liquid media containing fetal calf serum and subsequently cultured in media substantially lacking said fetal calf serum, with said culture lacking fetal calf serum used for production of a therapeutic product.

32. The method of claim 1, wherein said contact between said cell and liquid media is between 1 minute to 96 hours.

33. The method of claim 1, wherein said contact between said cell and liquid media is between 12 hours to 72 hours.

34. The method of claim 1, wherein said contact between said cell and liquid media is between 24 hours to 48 hours.

35. The method of claim 1, wherein said contact between said cell and liquid media is approximately 24 hours.

36. The method of claim 1, wherein said contact between said cell and liquid media is selected for a timepoint in which optimal secretion of therapeutic factors occurs in said liquid media.

37. The method of claim 1, wherein said liquid media is administered via a route of administration selected from: subcutaneously, intramuscularly, intra-ocularly, intravenously, topically, sublingually, or intralymphatically.

38. The method of claim 1, wherein said liquid media is administered intramuscularly.

39. The method of claim 1, wherein said liquid media is concentrated and desalted prior to administration.
